(19) United States
(12) Patent Application Publication
(10) Pub. No.: US 2026/0087710 A1
(43) Pub. Date: Mar. 26, 2026

(54) GENERATION OF ARTIFICIAL CONTRAST-ENHANCED RADIOLOGICAL IMAGES

(71) Applicant: BAYER AKTIENGESELLSCHAFT, Leverkusen (DE)

(72) Inventors: Felix Karl KREIS, Leverkusen (DE); Ivo Matteo BALTRUSCHAT, Leverkusen (DE); Matthias LENGA, Leverkusen (DE); Gregor JOST, Leverkusen (DE); Gunnar SCHÜTZ, Leverkusen (DE); Gesine KNOBLOCH, Leverkusen (DE); Christian LIENERTH, Leverkusen (DE); Hubertus PIETSCH, Leverkusen (DE)

(21) Appl. No.: 19/108,637

(22) PCT Filed: Aug. 29, 2023
(86) PCT No.: PCT/EP2023/073596

§ 371 Date: Mar. 4, 2025

(30) Foreign Application Priority Data
Sep. 5, 2022 (EP) 22193834.3
Oct. 21, 2022 (EP) 22202951.4
Feb. 24, 2023 (EP) 23158614.0
Apr. 19, 2023 (EP) 23168725.2

(51) Int. Cl. G06T 11/00; A61B 6/00; A61K 49/10; G06T 5/50

(52) U.S. Cl. CPC G06T 12/30; A61B 6/481; A61K 49/108; G06T 5/50; G06T 2207/10081; G06T 2207/10088; G06T 2207/20224; G06T 2207/30004; G06T 2210/41

(57) ABSTRACT

The present invention relates to the technical field of generating artificial contrast-enhanced radiological images.

The present disclosure is concerned with the technical field of generation of artificial contrast-enhanced radiological images.

WO2019/074938A1 discloses a method for reducing the amount of contrast agent in the generation of radiological images with the aid of an artificial neural network.

In the disclosed method, a training data set is created in a first step. The training data set comprises, for a large number of persons and for each person, i) a native radiological image (zero-contrast image), ii) a radiological image after administration of a low amount of contrast agent (low-contrast image) and iii) a radiological image after administration of a standard amount of contrast agent (full-contrast image).

In a second step, an artificial neural network is trained to predict for each person of the training data set, on the basis of the native image and the image after administration of a low amount of contrast agent, an artificial radiological image showing an acquisition region after administration of the standard amount of contrast agent. The measured radiological image after administration of a standard amount of contrast agent serves in each case as reference (ground truth) in the training.

In a third step, the trained artificial neural network can be used to predict for a new person, on the basis of a native image and of a radiological image after administration of a low amount of contrast agent, an artificial radiological image which shows the acquired region as it would look if a standard amount of contrast agent had been administered.

The method disclosed in WO2019/074938A1 has disadvantages.

For instance, training data are required for training the artificial neural network. A large number of radiological examinations need to be carried out on a large number of persons and the training data need to be generated in order to be able to train the network.

The artificial neural network disclosed in WO2019/074938A1 is trained to predict a radiological image after administration of a standard amount of a contrast agent. The artificial neural network is not configured and not trained to predict a radiological image after administration of an amount lower or higher than the standard amount of contrast agent. The method described in WO2019/074938A1 can in principle be trained to predict a radiological image after administration of an amount of contrast agent different than the standard amount, but this requires further training data and further training.

It would be desirable to be able to generate radiological images with variable contrast enhancement without needing to generate training data for each individual contrast enhancement and without needing to train an artificial neural network. It would additionally be desirable to be able to generate radiological images with variable contrast enhancement using a trackable deterministic process to generate the variable contrast enhancement. This facilitates the approval and use of a corresponding medical procedure, while minimizing false negative and false positive results. Machine learning methods employ statistical models, the generalizability of which is limited because they are usually based on a limited selection of training data. It would additionally be desirable to be able to generate radiological images with variable contrast enhancement using a wide variety of contrast agents. It would additionally be desirable to be able to use the method for generating radiological images with variable contrast enhancement using a wide variety of different contrast agents irrespective of their physical, chemical, physiological or other properties.

These and other objects are achieved by the subject matter of the independent claims. Preferred embodiments of the present disclosure are found in the dependent claims, in the present description and in the drawings.

The present invention thus provides in a first aspect a computer-implemented method for generating a synthetic contrast-enhanced radiological image, comprising the steps of:
receiving or generating a first representation, where the first representation represents an examination region of an examination object without contrast agent or after administration of a first amount of a contrast agent in frequency space or in real space,
receiving or generating a second representation, where the second representation represents the examination region of the examination object after an administration of a second amount of the contrast agent in frequency space or in real space,
generating a third representation based on the first representation and the second representation, where the generation of the third representation comprises a subtraction of the first representation from the second representation,
optionally: generating a weighted third representation, where the generation of the weighted third representation comprises a frequency-dependent weighting of the third representation,
generating a fourth representation, where the generation of the fourth representation comprises an α-fold addition of the optionally weighted third representation to the first representation or to the second representation, where a is a positive or negative real number,
if the fourth representation represents the examination region in frequency space: transforming the fourth representation into a fourth representation of the examination region in real space,
outputting and/or storing the fourth representation of the examination region in real space and/or transmitting the fourth representation of the examination region in real space to a separate computer system.

The present disclosure further provides a computer system comprising:
a processor; and
a memory that stores an application program configured to perform an operation when executed by the processor, said operation comprising:
   receiving or generating a first representation, where the first representation represents an examination region of an examination object without contrast agent or after administration of a first amount of a contrast agent in frequency space or in real space,
   receiving or generating a second representation, where the second representation represents the examination region of the examination object after an administration of a second amount of the contrast agent in frequency space or in real space,
   generating a third representation based on the first representation and the second representation, where the generation of the third representation comprises a subtraction of the first representation from the second representation,
   optionally: generating a weighted third representation, where the generation of the weighted third representation comprises a frequency-dependent weighting of the third representation,
   generating a fourth representation, where the generation of the fourth representation comprises an α-fold addition of the optionally weighted third representation to the first representation or to the second representation, where a is a positive or negative real number,
   if the fourth representation represents the examination region in frequency space: transforming the fourth representation into a fourth representation of the examination region in real space,
   outputting and/or storing the fourth representation of the examination region in real space and/or transmitting the fourth representation of the examination region in real space to a separate computer system.

The present disclosure further provides a computer program that can be loaded into a working memory of a computer system, where it causes the computer system to execute the following steps:
receiving or generating a first representation, where the first representation represents an examination region of an examination object without contrast agent or after administration of a first amount of a contrast agent in frequency space or in real space,
receiving or generating a second representation, where the second representation represents the examination region of the examination object after an administration of a second amount of the contrast agent in frequency space or in real space,
generating a third representation based on the first representation and the second representation, where the generation of the third representation comprises a subtraction of the first representation from the second representation,
optionally: generating a weighted third representation, where the generation of the weighted third representation comprises a frequency-dependent weighting of the third representation,
generating a fourth representation, where the generation of the fourth representation comprises an α-fold addition of the optionally weighted third representation to the first representation or to the second representation, where α is a positive or negative real number,
if the fourth representation represents the examination region in frequency space: transforming the fourth representation into a fourth representation of the examination region in real space,
outputting and/or storing the fourth representation of the examination region in real space and/or transmitting the fourth representation of the examination region in real space to a separate computer system.

The present disclosure further provides for the use of a contrast agent in a radiological examination method comprising:
receiving or generating a first representation, where the first representation represents an examination region of an examination object without contrast agent or after administration of a first amount of the contrast agent in frequency space or in real space,
receiving or generating a second representation, where the second representation represents the examination region of the examination object after an administration of a second amount of the contrast agent in frequency space or in real space,
generating a third representation based on the first representation and the second representation, where the generation of the third representation comprises a subtraction of the first representation from the second representation,
optionally: generating a weighted third representation, where the generation of the weighted third representation comprises a frequency-dependent weighting of the third representation,
generating a fourth representation, where the generation of the fourth representation comprises an α-fold addition of the optionally weighted third representation to the first representation or to the second representation, where α is a positive or negative real number,
if the fourth representation represents the examination region in frequency space: transforming the fourth representation into a fourth representation of the examination region in real space,
outputting and/or storing the fourth representation of the examination region in real space and/or transmitting the fourth representation of the examination region in real space to a separate computer system.

The present disclosure further provides a contrast agent for use in a radiological examination method comprising:
receiving or generating a first representation, where the first representation represents an examination region of an examination object without contrast agent or after administration of a first amount of the contrast agent in frequency space or in real space,
receiving or generating a second representation, where the second representation represents the examination region of the examination object after an administration of a second amount of the contrast agent in frequency space or in real space,
generating a third representation based on the first representation and the second representation, where the generation of the third representation comprises a subtraction of the first representation from the second representation,
optionally: generating a weighted third representation, where the generation of the weighted third representation comprises a frequency-dependent weighting of the third representation,
generating a fourth representation, where the generation of the fourth representation comprises an α-fold addition of the optionally weighted third representation to the first representation or to the second representation, where α is a positive or negative real number,
if the fourth representation represents the examination region in frequency space: transforming the fourth representation into a fourth representation of the examination region in real space,
outputting and/or storing the fourth representation of the examination region in real space and/or transmitting the fourth representation of the examination region in real space to a separate computer system.

The present disclosure further provides a kit comprising a computer program product and a contrast agent, where the computer program product comprises a computer program that can be loaded into a working memory of a computer system, where it causes the computer system to execute the following steps:
receiving or generating a first representation, where the first representation represents an examination region of an examination object without contrast agent or after administration of a first amount of the contrast agent in frequency space or in real space,
receiving or generating a second representation, where the second representation represents the examination region of the examination object after an administration of a second amount of the contrast agent in frequency space or in real space,
generating a third representation based on the first representation and the second representation, where the generation of the third representation comprises a subtraction of the first representation from the second representation,
optionally: generating a weighted third representation, where the generation of the weighted third representation comprises a frequency-dependent weighting of the third representation,
generating a fourth representation, where the generation of the fourth representation comprises an α-fold addition of the optionally weighted third representation to the first representation or to the second representation, where α is a positive or negative real number,
if the fourth representation represents the examination region in frequency space: transforming the fourth representation into a fourth representation of the examination region in real space,
outputting and/or storing the fourth representation of the examination region in real space and/or transmitting the fourth representation of the examination region in real space to a separate computer system.

The subjects of the present disclosure will be more particularly elucidated below, without distinguishing between the subjects (method, computer system, computer program (product), use, contrast agent for use, kit). Rather, the elucidations that follow are intended to apply by analogy to all subjects, irrespective of the context (method, computer system, computer program (product), use, contrast agent for use, kit) in which they occur.

Where steps are stated in an order in the present description or in the claims, this does not necessarily mean that this disclosure is limited to the order stated. Instead, it is conceivable that the steps are also executed in a different order or else in parallel with one another, the exception being when one step builds on another step, thereby making it imperative that the step building on the previous step be executed next (which will however become clear in the individual case). The stated orders thus constitute preferred embodiments.

In certain places, the invention will be more particularly elucidated with reference to drawings. The drawings show specific embodiments having specific features and combinations of features, which are intended primarily for illustrative purposes; the invention is not to be understood as being limited to the features and combinations of features shown in the drawings. Furthermore, statements made in the description of the drawings in relation to features and combinations of features are intended to be generally applicable, that is to say applicable to other embodiments too and not limited to the embodiments shown.

The present disclosure describes means by which one or more artificial radiological images are generated on the basis of at least two representations representing an examination region of an examination object after addition/administration/use of varying amounts of contrast agent, in which the contrast between regions with contrast agent and regions without contrast agent can be varied.

The “examination object” is normally a living being, preferably a mammal, most preferably a human.

The “examination region” is a part of the examination object, for example an organ or part of an organ or a plurality of organs or another part of the examination object.

For example, the examination region may be a liver, kidney, heart, lung, brain, stomach, bladder, prostate, intestine or a part thereof or another part of the body of a mammal (for example a human).

In one embodiment, the examination region includes a liver or part of a liver or the examination region is a liver or part of a liver of a mammal, preferably a human.

In a further embodiment, the examination region includes a brain or part of a brain or the examination region is a brain or part of a brain of a mammal, preferably a human.

In a further embodiment, the examination region includes a heart or part of a heart or the examination region is a heart or part of a heart of a mammal, preferably a human.

In a further embodiment, the examination region includes a thorax or part of a thorax or the examination region is a thorax or part of a thorax of a mammal, preferably a human.

In a further embodiment, the examination region includes a stomach or part of a stomach or the examination region is a stomach or part of a stomach of a mammal, preferably a human.

In a further embodiment, the examination region includes a pancreas or part of a pancreas or the examination region is a pancreas or part of a pancreas of a mammal, preferably a human.

In a further embodiment, the examination region includes a kidney or part of a kidney or the examination region is a kidney or part of a kidney of a mammal, preferably a human.

In a further embodiment, the examination region includes one or both lungs or part of a lung of a mammal, preferably a human.

In a further embodiment, the examination region includes a breast or part of a breast or the examination region is a breast or part of a breast of a female mammal, preferably a female human.

In a further embodiment, the examination region includes a prostate or part of a prostate or the examination region is a prostate or part of a prostate of a male mammal, preferably a male human.

The examination region, also referred to as the field of view (FOV), is in particular a volume that is imaged in radiological images. The examination region is typically defined by a radiologist, for example on a localizer image. It is of course also possible for the examination region to be alternatively or additionally defined in an automated manner, for example on the basis of a selected protocol.

The examination region is subjected to a radiological examination.

“Radiology” is the branch of medicine that is concerned with the use of electromagnetic rays and mechanical waves (including for instance ultrasound diagnostics) for diagnostic, therapeutic and/or scientific purposes. Besides X-rays, other ionizing radiation such as gamma radiation or electrons are also used. Imaging being a key application, other imaging methods such as sonography and magnetic resonance imaging (nuclear magnetic resonance imaging) are also counted as radiology, even though no ionizing radiation is used in these methods. The term “radiology” in the context of the present disclosure thus encompasses in particular the following examination methods: computed tomography, magnetic resonance imaging, sonography.

In one embodiment of the present disclosure, the radiological examination is a magnetic resonance imaging examination.

In a further embodiment, the radiological examination is a computed tomography examination.

In a further embodiment, the radiological examination is an ultrasound examination.

In radiological examinations, contrast agents are commonly used for contrast enhancement.

“Contrast agents” are substances or mixtures of substances that improve the depiction of structures and functions of the body in radiological examinations.

In computed tomography, iodine-containing solutions are normally used as contrast agents. In magnetic resonance imaging (MRI), superparamagnetic substances (e.g. iron oxide nanoparticles, superparamagnetic iron-platinum particles (SIPPs)) or paramagnetic substances (e.g. gadolinium chelates, manganese chelates) are usually used as contrast agents. In the case of sonography, liquids containing gas-filled microbubbles are normally administered intravenously. Examples of contrast agents can be found in the literature (see for example A. S. L. Jascinth et al.: Contrast Agents in computed tomography: A Review, Journal of Applied Dental and Medical Sciences, 2016, vol. 2, issue 2, 143-149; H. Lusic et al.: X-ray-Computed Tomography Contrast Agents, Chem. Rev. 2013, 113, 3, 1641-1666; https://www.radiology.wisc.edu/wp-content/uploads/2017/10/contrast-agents-tutorial.pdf, M. R. Nouh et al.: Radiographic and magnetic resonances contrast agents: Essentials and tips for safe practices, World J Radiol. 2017 Sep. 28; 9(9): 339-349; L. C. Abonyi et al.: Intravascular Contrast Media in Radiography: Historical Development & Review of Risk Factors for Adverse Reactions, South American Journal of Clinical Research, 2016, vol. 3, issue 1,1-10; ACR Manual on Contrast Media, 2020, ISBN: 978-1-55903-012-0; A. Ignee et al.: Ultrasound contrast agents, Endosc Ultrasound. 2016 November-December; 5(6): 355-362).

MRI contrast agents exert their effect in an MRI examination by altering the relaxation times of structures that take up contrast agents. A distinction can be made between two groups of substances: paramagnetic and superparamagnetic substances. Both groups of substances have unpaired electrons that induce a magnetic field around the individual atoms or molecules. Superparamagnetic contrast agents result in a predominant shortening of T2, whereas paramagnetic contrast agents mainly result in a shortening of T1. The effect of said contrast agents is indirect, since the contrast agent does not itself emit a signal, but instead merely influences the intensity of signals in its vicinity. An example of a superparamagnetic contrast agent is iron oxide nanoparticles (SPIO, superparamagnetic iron oxide). Examples of paramagnetic contrast agents are gadolinium chelates such as gadopentetate dimeglumine (trade name: Magnevist® and others), gadoteric acid (Dotarem®, Dotagita®, Cyclolux®), gadodiamide (Omniscan®), gadoteridol (ProHance®), gadobutrol (Gadovist®), gadopiclenol (Elucirem, Vueway) and gadoxetic acid (Primovist®/Eovist®).

In one embodiment, the radiological examination is an MRI examination in which an MRI contrast agent is used.

In a further embodiment, the radiological examination is a CT examination in which a CT contrast agent is used.

In a further embodiment, the radiological examination is a CT examination in which an MRI contrast agent is used.

The generation of an artificial radiological image with variable contrast enhancement is based on at least two representations of the examination region, a first representation and a second representation.

The first representation and the second representation are the result of a radiological examination. The first representation and the second representation are preferably measured radiological images or have been generated on the basis of measured radiological images. The first representation and/or the second representation may each be an MRI image, a CT image, an ultrasound image and/or another radiological image.

The first representation represents the examination region without contrast agent or after administration of a first amount of a contrast agent. Preferably, the first representation represents the examination region without contrast agent.

The second representation represents the examination region after administration of a second amount of a contrast agent. The second amount is larger than the first amount (which, as described, may also be zero). The expression “after the second amount of a contrast agent” should not be understood as meaning that the first amount and the second amount add up in the examination region (unless the first amount is zero). Thus, the expression “the representation represents the examination region after administration of a (first or second) amount” should rather be understood as meaning: “the representation represents the examination region with a (first or second) amount” or “the representation represents the examination region including a (first or second) amount”.

In one embodiment, both the first amount and the second amount of the contrast agent are smaller than the standard amount.

In a further embodiment, the second amount of the contrast agent corresponds to the standard amount.

In a further embodiment, the first amount of the contrast agent is equal to zero and the second amount of the contrast agent is smaller than the standard amount.

In a further embodiment, the first amount of the contrast agent is equal to zero and the second amount of the contrast agent corresponds to the standard amount.

The standard amount is normally the amount recommended by the manufacturer and/or distributor of the contrast agent and/or the amount authorized by a regulatory authority and/or the amount specified in a package leaflet for the contrast agent.

For example, the standard amount of Primovist® is 0.025 mmol Gd-EOB-DTPA disodium/kg body weight.

In one embodiment of the present disclosure, the contrast agent is an agent that includes gadolinium(III) 2-[4,7,10-tris(carboxymethyl)-1,4,7,10-tetrazacyclododec-1-yl]acetic acid (also referred to as gadolinium-DOTA or gadoteric acid).

In a further embodiment, the contrast agent is an agent that includes gadolinium(III) ethoxybenzyldiethylenetriaminepentaacetic acid (Gd-EOB-DTPA); preferably, the contrast agent includes the disodium salt of gadolinium(III) ethoxybenzyldiethylenetriaminepentaacetic acid (also referred to as gadoxetic acid).

In one embodiment of the present disclosure, the contrast agent is an agent that includes gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate (also referred to as gadopiclenol) (see for example WO2007/042504 and WO2020/030618 and/or WO2022/013454).

In one embodiment of the present disclosure, the contrast agent is an agent that includes dihydrogen [(±)-4-carboxy-5,8,11-tris(carboxymethyl)-1-phenyl-2-oxa-5,8,11-triazatridecan-13-oato(5-)]gadolinate(2−) (also referred to as gadobenic acid).

In one embodiment of the present disclosure, the contrast agent is an agent that includes tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate (also referred to as gadoquatrane) (see for example J. Lohrke et al.: Preclinical Profile of Gadoquatrane: A Novel Tetrameric, Macrocyclic High Relaxivity Gadolinium-Based Contrast Agent. Invest Radiol., 2022, 1, 57(10): 629-638; WO2016193190).

In one embodiment of the present disclosure, the contrast agent is an agent that comprises a Gd³⁺ complex of a compound of the formula (I)

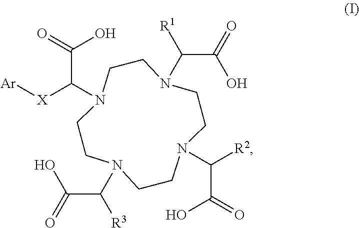

where
Ar is a group selected from

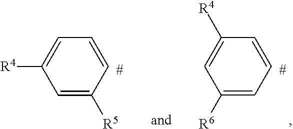

where # is the linkage to X,
X is a group selected from
CH₂, (CH₂)₂, (CH₂)₃, (CH₂)₄ and *—(CH₂)₂—O—CH₂—#,
where * is the linkage to Ar and # is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof.

In one embodiment of the present disclosure, the contrast agent is an agent that comprises a Gd³⁺ complex of a compound of the formula (II)

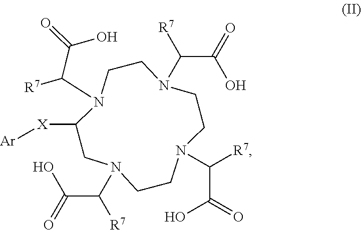

where
Ar is a group selected from

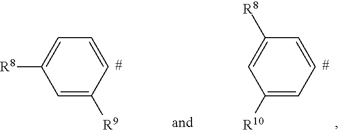

where # is the linkage to X,
X is a group selected from CH₂, (CH₂)₂, (CH₂)₃, (CH₂)₄ and *—(CH₂)₂—O—CH₂—#, where * is the linkage to Ar and # is the linkage to the acetic acid residue,
R⁷ is a hydrogen atom or a group selected from C₁-C₃ alkyl, —CH₂OH, —(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and
(H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

The term “C₁-C₃ alkyl” denotes a linear or branched, saturated monovalent hydrocarbon group having 1, 2 or 3 carbon atoms, for example methyl, ethyl, n-propyl or isopropyl. The term “C₂-C₄ alkyl” denotes a linear or branched, saturated monovalent hydrocarbon group having 2, 3 or 4 carbon atoms.

The term “C₂-C₄ alkoxy” denotes a linear or branched, saturated monovalent group of the formula (C₂-C₄ alkyl)-O—, in which the term “C₂-C₄ alkyl” is as defined above, for example a methoxy, ethoxy, n-propoxy or isopropoxy group.

In one embodiment of the present disclosure, the contrast agent is an agent that includes gadolinium 2,2′,2″-(10-{1-carboxy-2-[2-(4-ethoxyphenyl)ethoxy]ethyl}-1,4,7,10-tetraazacyclododecane-1,4,7-triyl)triacetate (see for example WO2022/194777, example 1).

In one embodiment of the present disclosure, the contrast agent is an agent that includes gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 2).

In one embodiment of the present disclosure, the contrast agent is an agent that includes gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 4).

In one embodiment of the present disclosure, the contrast agent is an agent that includes gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate) (see for example WO2022/194777, example 15).

In one embodiment of the present disclosure, the contrast agent is an agent that includes gadolinium 2,2′,2″-{10-[(1 S)-4-(4-butoxyphenyl)-1-carboxybutyl]-1,4,7,10-tetraazacyclododecane-1,4,7-triyl}triacetate (see for example WO2022/194777, example 31).

In one embodiment of the present disclosure, the contrast agent is an agent that includes gadolinium 2,2′,2″-{(2S)-10-(carboxymethyl)-2-[4-(2-ethoxyethoxy)benzyl]-1,4,7,10-tetraazacyclododecane-1,4,7-triyl}triacetate.

In one embodiment of the present disclosure, the contrast agent is an agent that includes gadolinium 2,2′,2″-[10-(carboxymethyl)-2-(4-ethoxybenzyl)-1,4,7,10-tetraazacyclododecane-1,4,7-triyl]triacetate.

In one embodiment of the present disclosure, the contrast agent is an agent that includes gadolinium(III) 5,8-bis(carboxylatomethyl)-2-[2-(methylamino)-2-oxoethyl]-10-oxo-2,5,8,11-tetraazadodecane-1-carboxylate hydrate (also referred to as gadodiamide).

In one embodiment of the present disclosure, the contrast agent is an agent that includes gadolinium(III) 2-[4-(2-hydroxypropyl)-7,10-bis(2-oxido-2-oxoethyl)-1,4,7,10-tetrazacyclododec-1-yl]acetate (also referred to as gadoteridol).

In one embodiment of the present disclosure, the contrast agent is an agent that includes gadolinium(III) 2,2′,2″-(10-((2R,3S)-1,3,4-trihydroxybutan-2-yl)-1,4,7,10-tetraazacyclododecane-1,4,7-triyl)triacetate (also referred to as gadobutrol or Gd-DO3A-butrol).

In a first step, the first representation and the second representation are received or generated.

The term “receiving” encompasses both the retrieving of representations and the accepting of representations transmitted for example to the computer system of the present disclosure. The representations may be received from a computed tomography system, from a magnetic resonance imaging system or from an ultrasound scanner. The radiological images may be read from one or more data memories and/or transmitted from a separate computer system.

The term “generating” preferably means that a representation is generated on the basis of another (for example a received) representation or on the basis of a plurality of other (for example received) representations. For example, a received representation may be a representation of an examination region of an examination object in real space. On the basis of this real-space representation it is possible for example to generate a representation of the examination region of the examination object in frequency space through a transform operation (for example a Fourier transform). Other ways of generating a representation on the basis of one or more other representations are described in this description.

The first representation and the second representation represent the examination region in real space or frequency space.

Radiological images resulting from radiological examinations are often obtained as representations in real space (also called image space).

The “real space” is the ordinary three-dimensional Euclidean space that corresponds to the space we humans experience with our senses and in which we move. A representation in real space is therefore the familiar representation for people.

In a representation in real space, also referred to in this description as real-space depiction or real-space representation, the examination region is normally represented by a large number of image elements (pixels or voxels) that may for example be in a raster arrangement, in which case each image element represents a part of the examination region and each image element may be assigned a color value or gray value. A format widely used in radiology for storing and processing representations in real space is the DICOM format. DICOM (Digital Imaging and Communications in Medicine) is an open standard for storing and exchanging information in medical image data management.

The “frequency space” is a domain in which a signal is considered to be the sum of its individual frequency components.

In a representation in frequency space, also referred to in this description as frequency-space depiction or frequency-space representation, the examination region is represented by a superposition of fundamental oscillations. For example, the examination region may be represented by a sum of sine and/or cosine functions having different amplitudes, frequencies and phases. The amplitudes and phases may be plotted as a function of the frequencies, for example, in a two- or three-dimensional representation. Normally, the lowest frequency (origin) is placed in the center. The further away from this center, the higher the frequencies. Each frequency can be assigned an amplitude, representing the frequency in the frequency-space depiction, and a phase indicating the extent of the shift of the respective oscillation with respect to a sine or cosine oscillation.

The raw data obtained in magnetic resonance imaging examinations (so-called k-space data) are an example of a representation in frequency space. Such raw data (k-space data) from magnetic resonance imaging examinations can be used directly as a first and/or second representation in the context of the present disclosure.

A representation in real space can for example be converted (transformed) by a Fourier transform operation into a representation in frequency space. Conversely, a representation in frequency space can for example be converted (transformed) by an inverse Fourier transform into a representation in real space.

Details about real-space depictions and frequency-space depictions and their respective interconversion are described in numerous publications, see for example https://see.stanford.edu/materials/1softaee261/book-fall-07.pdf.

It is possible to carry out a co-registration of representations in real space before they are converted into frequency-space depictions. “Co-registration” (also known in the prior art as “image registration”) is employed to bring two or more real-space depictions of the same examination region into the best possible conformity with one another. One of the real-space depictions is defined as the reference image, the other is termed the object image. In order to optimally fit this to the reference image, a compensating transform operation is calculated.

It is also possible to co-register representations in frequency space; it should be noted here that a translation in real space constitutes an additive linear phase ramp in frequency space. Scaling and rotation are on the other hand retained in the Fourier and inverse Fourier transform—scaling and rotation in frequency space is also scaling and rotation in real space (see for example S. Skare: Rigid Body Image Realignment in Image Space vs. k-Space, ISMRM SCIENTIFIC WORKSHOP on Motion Correction, 2014, https://cds.ismrm.org/protected/Motion_14/Program/Syllabus/Skare.pdf).

It should be noted that co-registration in frequency space does not have to be very precise, since high frequencies, which map image details and thus inaccuracies in the registration, are attenuated by the frequency filter. This is an advantage of the approach described in this disclosure over approaches in which operations are executed in real space.

On the basis of the first representation and the second representation, a third representation is generated.

The third representation represents the examination region in real space or in frequency space.

The third representation represents the signal enhancement (contrast agent signal representation) brought about in the examination region by the second amount of contrast agent. In other words, the third representation comprises the differences in the second representation compared to the first representation that are brought about by the second amount of contrast agent.

In a preferred embodiment, the generation of the third representation comprises a subtraction of the first representation from the second representation. In other words, in a preferred embodiment the third representation is the difference of the first and the second representation.

The subtraction may be carried out in real space or in frequency space.

When the first representation and the second representation represent the examination region in real space, then the subtraction of the first representation from the second representation is preferably carried out in real space; the result is a third representation (a contrast agent signal representation) in real space.

When the first representation and the second representation represent the examination region in frequency space, then the subtraction of the first representation from the second representation is preferably carried out in frequency space; the result is a third representation (a contrast agent signal representation) in frequency space.

However, it is also possible to first convert a first representation in real space and a second representation in real space into a first representation in frequency space and a second representation in frequency space, in order to then generate a third representation in frequency space by subtracting the first frequency-space representation from the second frequency-space representation.

Likewise, it is possible to first convert a first representation in frequency space and a second representation in frequency space into a first representation in real space and a second representation in real space, in order to then generate a third representation in real space by subtracting the first real-space representation from the second real-space representation.

Preferably, the generation of the third representation in frequency space is based on a first representation in frequency space and a second representation in frequency space. In such a third frequency-space representation, each frequency is represented by an amplitude value where the more strongly the frequency is influenced by the second amount of contrast agent, the higher this value.

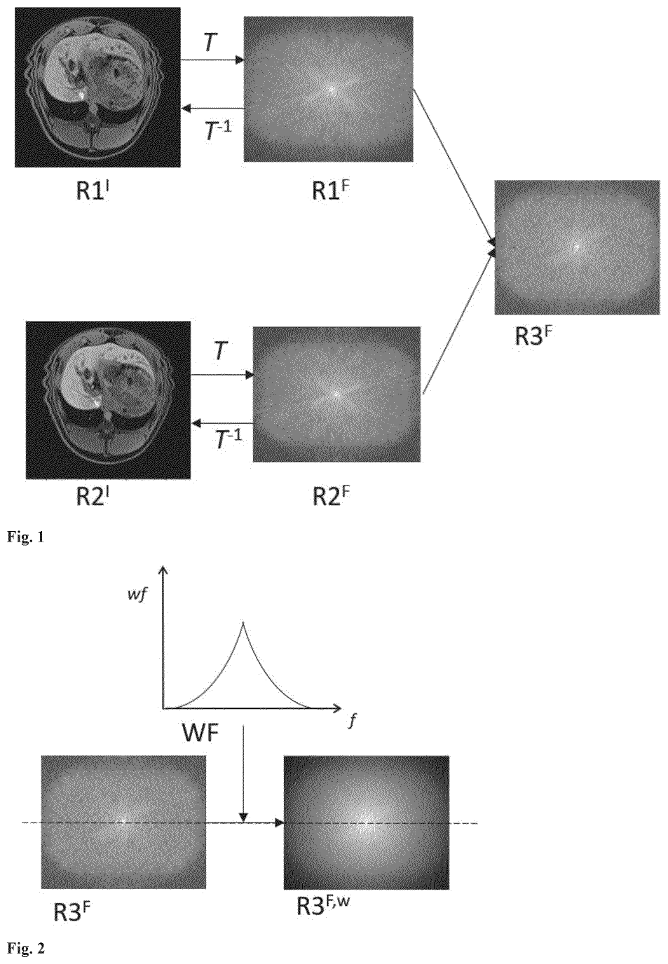

FIG. 1 shows by way of example and in schematic form an embodiment of the generation of a third representation based on a first representation and a second representation.

FIG. 1 shows an examination region of an examination object in the form of various representations.

A first representation R₁^{I} represents the examination region in real space without contrast agent or after administration of a first amount of a contrast agent. The examination region shown in FIG. 1 includes a liver of a pig. The first representation R₁^{I} is a magnetic resonance image.

The first real-space representation R₁^{I} can be converted into a first representation R1^{F} of the examination region in frequency space through a transform operation T, for example a Fourier transform. The first frequency-space representation R1^{F} represents the same examination region of the same examination object as the first real-space representation R₁^{I}, likewise without contrast agent or after administration of the first amount of the contrast agent.

The first frequency-space representation R1^{F} can be converted into the first real-space representation R₁^{I} by means of an inverse transform operation T¹. The inverse transform operation T¹ is the inverse transform of transform operation T.

A second representation R₂^{I} represents the same examination region of the same examination object as the first representation R₁^{I} in real space. The second real-space representation R₂^{I} represents the examination region after administration of a second amount of the contrast agent. The second amount is larger than the first amount (which, as described, may also be zero). The second representation R₁^{I} is likewise a magnetic resonance image. The contrast agent used in the example shown in FIG. 1 was a hepatobiliary MRI contrast agent. A hepatobiliary contrast agent has the characteristic features of being specifically taken up by liver cells (hepatocytes), accumulating in the functional tissue (parenchyma) and enhancing contrast in healthy liver tissue. An example of a hepatobiliary contrast agent is the disodium salt of gadoxetic acid (Gd-EOB-DTPA disodium), which is described in U.S. Pat. No. 6,039,931A and is commercially available under the trade names Primovist® and Eovist®. Further hepatobiliary contrast agents are described inter alia in WO2022/194777.

In the second real-space representation R₂^{I}, the contrast between the liver tissue and the surrounding tissue is enhanced as a result of the second amount of the contrast agent.

The second real-space representation R₂^{I} can be converted into a second representation R₂^{F} of the examination region in frequency space by means of the transform operation T. The second frequency-space representation R₂^{F} represents the same examination region of the same examination object as the second real-space representation R₂^{I}, likewise after administration of the second amount of the contrast agent.

The second frequency-space representation R₂^{F} can be converted into the second real-space representation R₂^{I} by means of the inverse transform operation T⁻¹.

On the basis of the first frequency-space representation R₁^{F} and the second frequency-space representation R₂^{F}, a third frequency-space representation R₃^{F} is generated. In the example shown in FIG. 1, the third frequency-space representation R₃^{F} is the difference of the second frequency-space representation R₂^{F} and the first frequency-space representation R₁^{F} (R₃^{F}=R₂^{F}−R₁^{I}).

The third frequency-space representation R₃^{F} may be subjected to a normalization, that is to say the amplitude values may be multiplied by a factor such that the amplitude having the highest value is represented for example by the hue “white” and the amplitude having the lowest value is represented for example by the hue “black”.

In such a normalization, it is also possible for negative values that can arise when subtracting the first representation from the second representation to be set to zero (or to another value) to avoid negative values.

The third frequency-space representation R₃^{F} represents the contrast enhancement brought about in the examination region by the second amount of the contrast agent.

In a further step, a weighted third representation can be generated based on the third representation. Such weighting of the third representation allows a higher weighting to be given to frequencies making a higher contribution to contrast than to frequencies making a smaller contribution to contrast. The expression “contrast” refers to the real-space depiction corresponding to the frequency-space depiction. Contrast information is represented in a frequency-space depiction by low frequencies, while the higher frequencies represent information about fine structures. Image noise is typically evenly distributed in the frequency depiction. The weighted third representation can thus be generated by applying a frequency-dependent weight function to the third representation in which low frequencies are given a higher weighting than high frequencies. The frequency-dependent weight function has the effect of a filter. The filter increases the signal-to-noise ratio by reducing the spectral noise density for high frequencies.

The weighting of the third representation (i.e. the generation of the weighted third representation) is done in frequency space. If the third representation is a real-space representation, it can be transformed into a third representation in frequency space by a Fourier transform operation.

The weighting of the third representation in frequency space can be done by multiplying the third representation in frequency space by a frequency-dependent weight function. In such a frequency-dependent weight function, each frequency is assigned a weight factor. If the weight factor of a particular frequency is for example zero, the amplitude of the corresponding frequency in the third representation will be set to zero when multiplying the third representation in frequency space by the frequency-dependent weight function, i.e. the frequency will be eliminated. If the weight factor of a particular frequency is for example 1, the amplitude of the corresponding frequency in the third representation will be unchanged when multiplying the third representation in frequency space by the frequency-dependent weight function, i.e. the frequency remains unchanged. If the weight factor of a particular frequency is for example 0.5, the amplitude of the corresponding frequency will be reduced to half its value when multiplying the third representation in frequency space by the frequency-dependent weight function, i.e. the corresponding frequency will be attenuated in the third representation in frequency space. If the weight factor of a particular frequency is for example 2, the amplitude of the corresponding frequency will be doubled when multiplying the third representation in frequency space by the frequency-dependent weight function, i.e. the corresponding frequency will be enhanced in the third representation in frequency space.

In the frequency-dependent weighting of the third representation, the amplitudes of the lower frequencies are preferably multiplied by a higher weight factor than the amplitudes of the higher frequencies. In a preferred embodiment, the higher the frequencies, the lower the weight factor by which amplitudes of frequencies are multiplied.

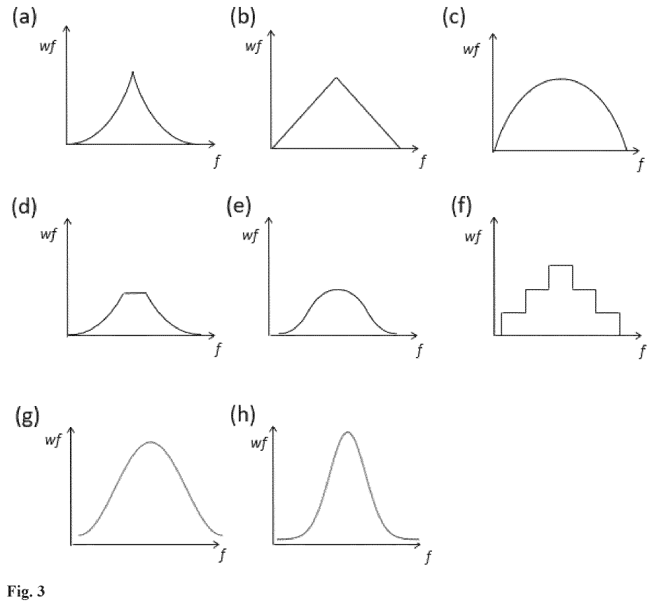

Examples of frequency-dependent weight functions are shown in FIG. 3.

FIG. 2 shows by way of example and in schematic form the generation of a weighted third representation R₃^{F,w}. FIG. 2 shows the third frequency-space representation R₃^{F} already shown in FIG. 1. The amplitude values of the third frequency-space representation R₃^{F} are multiplied by weight factors. The weight factors are frequency dependent, i.e. the weight factors are a function of the frequency. For reasons of illustration, the weight function WF is shown in FIG. 2 in two-dimensional form. The weight function WF shows the weight factors wf as a function of the frequency f along one dimension (along the dashed line). Along the dimension perpendicular to the dashed line in the same image plane, the weight function has the same shape; it is merely compressed, because the representation R₃^{F} in the present example is rectangular but not square.

The WF weight function multiplies the amplitudes of low frequencies (in the example shown, the frequencies increase outward from the center of the representation R₃^{F}) by a higher weight factor than the amplitudes of higher frequencies (which are further away from the center of the representation R₃^{F}); i.e. the low frequencies are given a higher weighting than the higher frequencies. This can be recognized in the weighted representation R₃^{F,w} in that the gray values toward the edges of the representation are darker than in the case of representation R₃^{F} and the overall brightness decreases outward from the center more rapidly than in the case of representation R₃^{F}.

The weighted representation R₃^{F,w} can be subjected to a normalization, that is to say the amplitude values can be multiplied by a factor such that the amplitude having the highest value is represented for example by the hue “white” and the amplitude having the lowest value is represented for example by the hue “black”.

FIG. 3 shows examples of frequency-dependent weight functions that can be used for weighting the third representation. For simplicity, the weight functions are represented as two-dimensional graphs in which the weight factors wf (ordinate) are plotted as a function of the frequency f (abscissa).

FIG. 3 (a) shows the weight function WF already shown in FIG. 2. In this weight function, the weight factors may for example decrease from the center exponentially as the frequency increases.

FIG. 3 (b) shows a weight function in which the weight factors decrease from the center linearly as the frequency increases.

FIG. 3 (c) shows a weight function in which the weight factors decrease from the center in the form of a reverse parabola as the frequency increases.

FIG. 3 (d) shows a weight function in which the weight factors are constant over a defined range about the center and then decrease exponentially from a threshold frequency.

FIG. 3 (e) shows a weight function in which the weight factors have the course of a cosine function about the center.

FIG. 3 (f) shows a weight function in which the weight factors have the course of a step function about the center.

FIG. 3 (g) shows a weight function in which the weight factors have the course of a Gaussian distribution function about the center.

FIG. 3 (h) shows a weight function in which the weight factors have the course of a Hann function about the center.

Combinations of the weight functions shown and further/other weight functions are possible. Examples of other weight functions can be found for example at https://de.wikipedia.org/wiki/Fensterfunktion#Beispiele_von_Fensterfunktionen; F. J. Harris et al.: On the Use of Windows for Harmonic Analysis with the Discrete Fourier Transform, Proceedings of the IEEE, vol. 66, No. 1, 1978; https://docs.scipy.org/doc/scipy/reference/signal.windows.html; K. M. M. Prabhu: Window Functions and Their Applications in Signal Processing, CRC Press, 2014, 978-1-4665-1583-3.

Weight functions that can be used are also referred to in the literature as window functions.

Preference is given to using weighting functions of proven utility for the weighting of k-space data in MRI imaging and spectroscopy, for example the Hann function (also referred to as the Hann window, see for example: Hanning Window, see for example R. Pohmann et al.: Accurate phosphorus metabolite images of the human heart by 3D acquisition-weighted CSI, Magnetic Resonance in Medicine: An Official Journal of the International Society for Magnetic Resonance in Medicine 45.5 (2001): 817-826).

Another preferred weight function is the Poisson function (Poisson window).

In a further step, a fourth representation is generated by combining the first representation with the optionally weighted third representation. Such a combination transfers to the first representation information about the contrast enhancement brought about by the second amount of the contrast agent in the examination region.

The combination can for example be or include an addition of the first representation and the optionally weighted third representation. Multiplicative combination or nonlinear combination is however also possible.

The generation of the fourth representation based on the optionally weighted third representation can be done in real space or in frequency space, i.e. an optionally weighted third frequency-space representation can be combined with a first frequency-space representation (for example by adding the optionally weighted third frequency-space representation to the first frequency-space representation a times), or an optionally weighted third real-space representation can be combined with a first real-space representation (for example by adding the optionally weighted third real-space representation to the first real-space representation a times).

It is in principle also possible to generate the fourth representation (in real space or in frequency space) by combining the optionally weighted third representation with the second representation (for example by adding the optionally weighted third representation to the second representation a times).

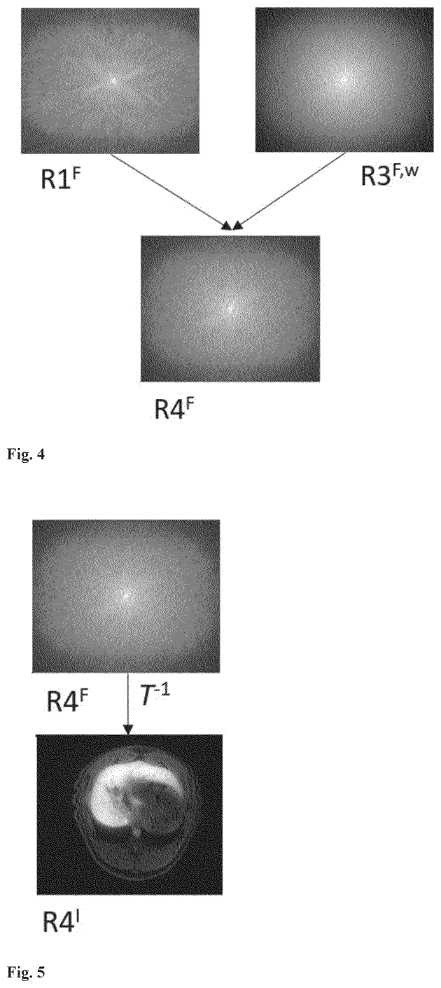

FIG. 4 shows by way of example and in schematic form an embodiment of the generation of a fourth representation. In FIG. 4, a fourth representation R₄^{F} of the examination region of the examination object in frequency space is generated by combining the first frequency-space representation R₁^{F} already shown in FIG. 1 with the weighted representation R₃^{F,w} already shown in FIG. 2. The combining in the example is done by addition. It is possible to subject the fourth representation R₄^{F} to a normalization.

If the fourth representation is a representation in frequency space (as shown in the example in FIG. 4), the fourth frequency-space representation is in a further step converted by means of a transform operation (for example an inverse Fourier transform) into a fourth real-space representation. If the fourth representation is a representation in real space (for example, because it had been generated by combining a first real-space representation with an optionally weighted third real-space representation), such a transform operation into real space is not required.

The fourth real-space depiction of the examination region can be output (for example displayed on a screen or printed using a printer), stored in a data memory and/or transmitted to a separate computer system.

FIG. 5 shows by way of example and in schematic form how a fourth representation R₄^{I} of the examination region in real space is generated from the fourth representation R₄^{F} of the examination region in frequency space already shown in FIG. 4 by means of the inverse transform operation T⁻¹ described with reference to FIG. 1.

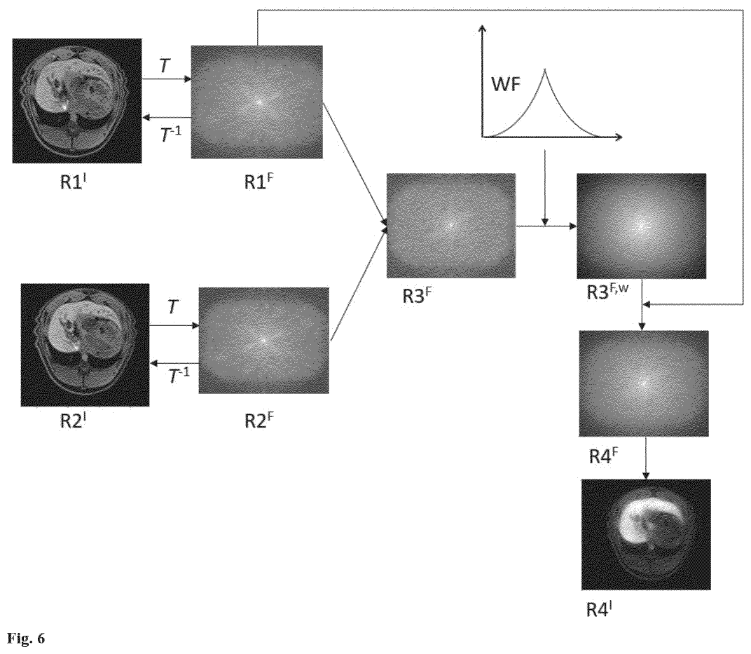

FIG. 6 shows by way of example and in schematic form the entire process as already shown in parts in FIG. 1, FIG. 2, FIG. 4, and FIG. 5.

When the third representation is generated by subtracting the first representation from the second representation, an addition of the (non-weighted) third representation to the first representation would again result in the second representation.

Addition of the weighted third representation to the first representation results in a different representation than the second representation.

The weighting allows the contrast information to be the focus, i.e. features in the resulting fourth representation are emphasized due to an increase in contrast brought about by the second amount of contrast agent.

It is possible to add the optionally weighted third representation to the first representation multiple times in order to achieve further contrast enhancement without amplifying interference and/or noise to the same degree as the contrast.

It is thus possible to add the optionally weighted third representation multiplied by a gain factor α to the first representation, where the gain factor α represents the degree to which the contrast in the fourth representation is increased. It is also possible here to opt for an enhancement of less than 1, i.e. the contrast between areas with contrast agent and areas without contrast agent is lower in the fourth representation than in the second representation. It is likewise possible to achieve an enhancement that is greater than the contrast enhancement brought about by a standard amount of contrast agent. With the method described in WO2019/074938A1, such a contrast enhancement is not possible without administering to people for the generation of training data an amount of contrast agent that is higher than the standard amount and thus outside the range approved by the regulatory authority.

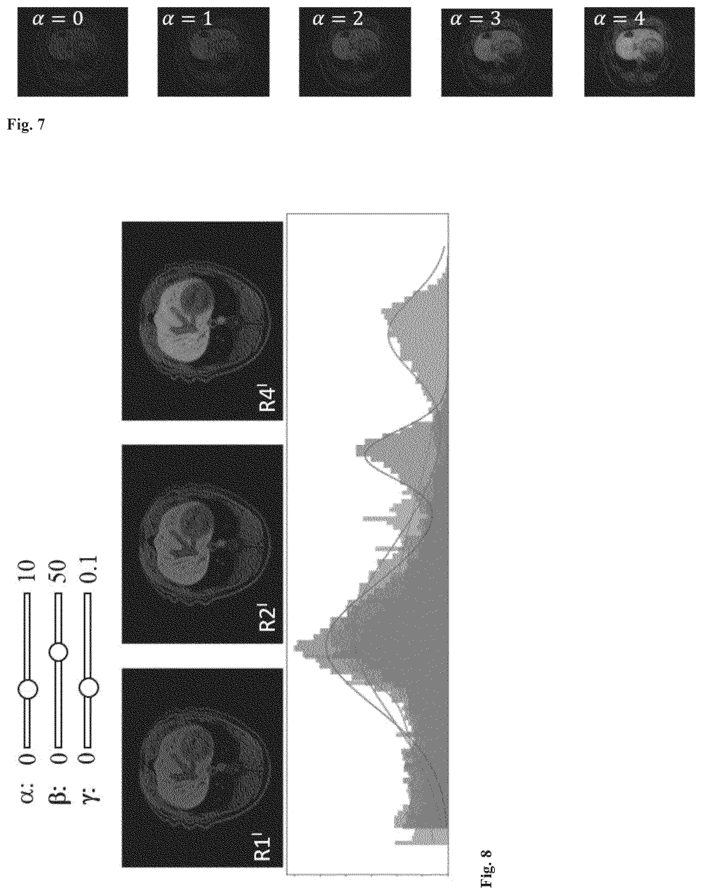

The gain factor α may be chosen by a user, i.e. it may be variable or predefined, i.e. predetermined. FIG. 7 shows by way of example and in schematic form various representations of an examination region of an examination object in real space. The representations differ by the gain factor α, which can assume values of 0, 1, 2, 3, and 4 in the present examples.

A gain factor of α=0 means that no contrast enhancement is performed in the first representation. The representation thus shows the original first representation in real space.

A gain factor of α=1 means that the optionally weighted third representation is added to the first representation once (in frequency space or in real space). The contrast enhancement is similar to the contrast enhancement in the second representation, but the weighting performed—for example weighting of low frequencies—results in it having less noise/interference.

A gain factor of α=2, 3 or 4 means that the optionally weighted third representation is added to the first representation twice, three times or four times (in frequency space or in real space). The contrast enhancement rises as the gain factor increases.

In the example shown in FIG. 7, an integer multiple of the optionally weighted third representation was in all cases added to the first representation. It is of course also possible to add a non-integer proportion of the optionally weighted third representation to the first representation (for example α=1.5 or α=3.7 or α=4.159). This means that an enhancement can be increased in a continuous manner.

Negative a values are also possible, which can for example be chosen so that regions of the examination region that experience a contrast agent-induced signal enhancement in the representation generated by measurement are completely dark (black) in the artificially generated representations.

The gain factor α is thus a positive or negative real number.

It is also possible to determine the gain factor α in an automated manner. “Automated” means without human assistance. It is for example possible to define at least one region in a real-space depiction of the first and/or second representation and/or to have it selected by a user and to set the gain factor α such that a gray value in the real-space depiction (or a different tonal value in the case of a depiction other than a gray-value depiction) assumes a defined value and/or is above or below a threshold value and/or that two gray values in two different selected or defined regions are a defined distance from one another and/or are at a distance from one another that is above or below a threshold value. It is also possible to employ other criteria in the automated determination of the gain factor α. The criteria for the automated determination of the gain factor α may for example be based on the histogram of the real-space depiction of the first, second, third, weighted third and/or fourth representation. Such a histogram may show the number of image elements having a defined tonal value or gray value.

FIG. 8 shows a preferred embodiment of an output of the artificial contrast-enhanced radiological image of an examination region by means of a computer system/computer program. The output is made to a user of the computer system and/or computer program of the present disclosure.

A first real-space representation R₁^{I} of an examination region of an examination object, a second real-space representation R₂^{I} of the examination region of the examination object and a fourth real-space representation R₄^{I} of the examination region of the examination object are displayed to the user (for example on a monitor).

The first representation R₁^{I} represents the examination region without contrast agent or after administration of a first amount of a contrast agent.

The second representation R₂^{I} represents the examination region after administration of a second amount of the contrast agent. The second amount is larger than the first amount.

The fourth representation R₄^{I} represents the examination region with enhanced contrast. The contrast between areas without contrast agent and areas with contrast agent is greater in the case of the fourth representation R₄^{I} than in the second representation R₂^{I}.

The fourth representation R₄^{I} was generated as described in this disclosure (see in particular the descriptions relating to FIG. 1 to FIG. 7).

All displayed representations are representations of the examination region in real space. In the example shown in FIG. 8, no frequency-space representations are displayed to the user. This is normally also not envisaged, since many users are unfamiliar with frequency-space representation.

Below the displayed representations R₁^{I}, R₂^{I}, and R₄^{I}, the histograms of the representations are displayed to the user in a superimposition.

Above the displayed representations R₁^{I}, R₂^{I}, and R₄^{I}, the user is provided with three virtual sliders with which the user can make adjustments. A first slider α allows the user to freely choose the gain factor α in a range from 1 to 10. The slider indicates that the gain factor can be increased from 1 to 10 in a continuous manner.

A second slider β and a third slider γ allow the user to alter the parameters of the weighting function. These parameters can for example determine how strongly weight factors decrease with increasing frequency.

The output shown in FIG. 8 is preferably configured so that the display of the fourth representation R₄^{I} is immediately updated when the user makes changes by means of one of the sliders. The user can then for example alter the gain factor α and immediately see how a change in the gain factor α affects the representation R₄^{I}. This allows the user to identify those settings that result in a fourth representation R₄^{I} of the examination region that is optimal for the user.

Any change to one of the parameters α, β and/or γ results in the computer system each time recalculating the fourth representation R₄^{I} on the basis of the altered parameter(s) and displaying this in the background. The same applies also to the histogram of the fourth representation R₄^{L}

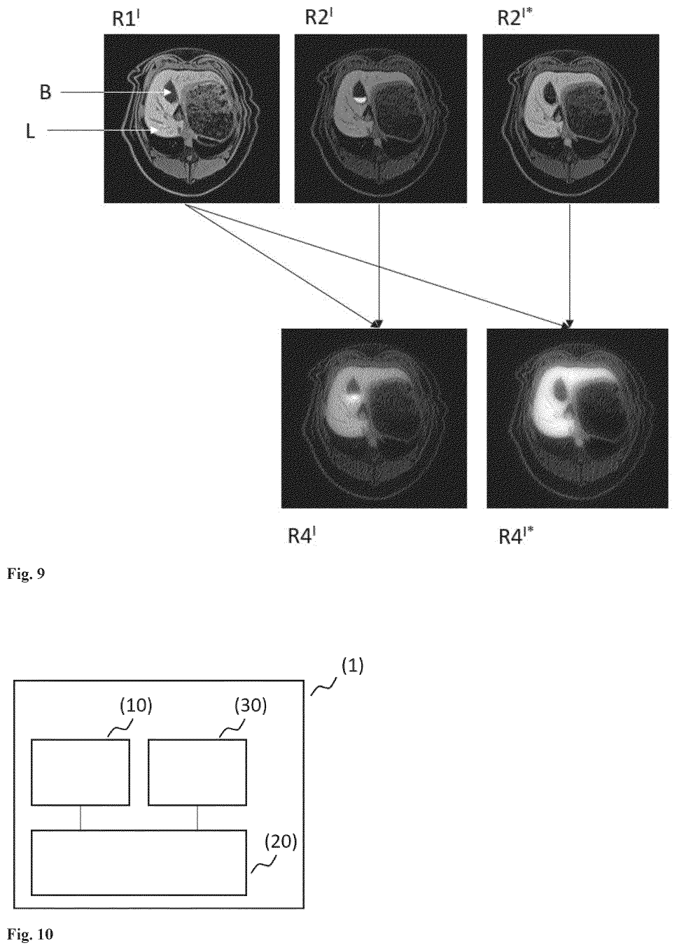

It is possible that the contrast enhancement according to the present disclosure up to now will bring about a contrast enhancement that is undesirable to the user. This shall be explained using an example. The example is depicted schematically in FIG. 9.

FIG. 9 shows a first representation R₁^{I} and a second representation R₂^{I} of an examination region of an examination object. The examination region includes the liver L and the gallbladder B of a pig. The first representation R₁^{I} represents the examination region without contrast agent or after administration of a first amount of a contrast agent in real space. The second representation R₂^{I} represents the examination region after administration of a second amount of the contrast agent in real space. The second amount is larger than the first amount. In the second representation R₂^{I} it can be seen that the gallbladder has partially filled, for example with a fluid comprising the contrast agent or another fluid, resulting in a high contrast between the partially filled gallbladder and the surrounding areas.

A contrast enhancement as described in the present disclosure results in the contrast between the partially filled gallbladder and the other areas being further enhanced in an artificial contrast-enhanced radiological image R₄^{I} of the examination region. It is however conceivable that a user will instead be interested in a contrast enhancement of the liver.

In a preferred embodiment, the computer system and the computer program of the present disclosure are configured to receive an input from the user. In the input, the user can specify one or more areas for which they do not want contrast enhancement. The user can draw such an area in the first, second and/or fourth representation in real space using, for example, a mouse or another input means. For instance, in the example shown in FIG. 9, the user can select and/or highlight the gallbladder in the first representation R₁^{I}, the second representation R₂^{I} and/or the fourth representation R₄^{I}. The computer system and the computer program may be configured to set the tonal values or gray values of all image elements (pixels, voxels) that represent the (highlighted) gallbladder to zero. The result is the representation R₂^{I*}, in which the gallbladder is represented by black image elements. If the contrast enhancement is performed on the basis of the representations R₁^{I} and R₂^{I*} (or on the basis of their corresponding frequency-space representations) as described in this disclosure, this results in the artificial contrast-enhanced radiological image R₄ⁱ*, in which there is now increased contrast particularly between the liver L and the other areas, but the partially filled gallbladder is no longer depicted with enhanced contrast.

In a preferred embodiment, areas that are not (to be) contrast enhanced are determined in an automated manner. Preferably, the quotient of the tonal values is determined for all pairs of corresponding image elements (i.e. having the same coordinates) of the first real-space representation R₁^{I} and the second real-space representation R₂^{I}:

$$Q = g 2 ( x , y , z ) / g 1 ( x , y , z )$$

where Q is a quotient of tonal values, g2(x, y, z) is the tonal value of the image element having the coordinates x, y, z in the second representation R₂^{I} and g1(x, y, z) is the tonal value of the image element having the same coordinates x, y, z in the first representation R₁^{I}. The quotient Q of the tonal values is a measure of how much brighter the image element having the coordinates x, y, z is depicted in the second representation compared to the corresponding image element in the first representation. It specifies the contrast enhancement brought about by the second amount of the contrast agent in the examination region represented by image elements having the coordinates x, y, z.

The computer system and the computer program may be configured to compare the quotients of the tonal values for all image elements with a predefined threshold value. The predefined threshold value specifies a maximum contrast enhancement to be expected due to the contrast agent.

If a quotient of the tonal values of corresponding image elements is greater than the predefined threshold value, the tonal values of the corresponding image elements can be set to zero.

FIG. 10 shows by way of example and in schematic form a computer system according to the present disclosure.

A “computer system” is an electronic data processing system that processes data by means of programmable calculation rules. Such a system typically comprises a “computer”, which is the unit that includes a processor for carrying out logic operations, and peripherals.

In computer technology, “peripherals” refers to all devices that are connected to the computer and are used for control of the computer and/or as input and output devices. Examples thereof are monitor (screen), printer, scanner, mouse, keyboard, drives, camera, microphone, speakers, etc. Internal ports and expansion cards are also regarded as peripherals in computer technology.

The computer system (1) shown in FIG. 10 comprises a receiving unit (10), a control and calculation unit (20) and an output unit (30).

The control and calculation unit (20) serves for control of the computer system (1), for coordination of the data flows between the units of the computer system (1), and for the performance of calculations.

The control and calculation unit (20) is configured:
to generate a first representation or to cause the receiving unit (10) to receive the first representation, where the first representation represents an examination region of an examination object without contrast agent or after administration of a first amount of a contrast agent in real space or in frequency space,
to generate a second representation or to cause the receiving unit (10) to receive the second representation, where the second representation represents the examination region of the examination object after an administration of a second amount of the contrast agent in real space or in frequency space,
to generate a third representation based on the first representation and the second representation, where the generation of the third representation comprises a subtraction of the first representation from the second representation, where the third representation represents the examination region in real space or in frequency space,
optionally to generate a weighted third representation based on the third representation, where the generation of the weighted third representation comprises a frequency-dependent weighting of the third representation in frequency space,
to generate a fourth representation based on the optionally weighted third representation and the first or second representation, where the generation of the fourth representation comprises an α-fold addition of the optional third representation to the first representation or to the second representation, where α is a positive or negative real number,
to transform the fourth representation into a representation of the examination region in real space, if the fourth representation represents the examination region in frequency space,
to cause the output unit (30) to output the fourth representation of the examination region in real space and/or to store it and/or to transmit it to a separate computer system.

FIG. 11 shows by way of example and in schematic form a further embodiment of the computer system. The computer system (1) comprises a processing unit (21) connected to a memory (22). The processing unit (21) and the memory (22) form a control and calculation unit, as shown in FIG. 10.

The processing unit (21) may comprise one or more processors alone or in combination with one or more memories. The processing unit (21) may be customary computer hardware that is able to process information such as digital images, computer programs and/or other digital information. The processing unit (21) normally consists of an arrangement of electronic circuits, some of which can be designed as an integrated circuit or as a plurality of integrated circuits connected to one another (an integrated circuit is sometimes also referred to as a “chip”). The processing unit (21) may be configured to execute computer programs that can be stored in a working memory of the processing unit (21) or in the memory (22) of the same or of a different computer system.

The memory (22) may be customary computer hardware that is able to store information such as digital images (for example representations of the examination region), data, computer programs and/or other digital information either temporarily and/or permanently. The memory (22) may comprise a volatile and/or nonvolatile memory and may be nonremovable or removable. Examples of suitable memories are RAM (random access memory), ROM (read-only memory), a hard disk, a flash memory, an exchangeable computer floppy disk, an optical disk, a magnetic tape or a combination of the aforementioned. Optical disks can include compact disks with read-only memory (CD-ROM), compact disks with read/write function (CD-R/W), DVDs, Blu-ray disks and the like.

The processing unit (21) may be connected not just to the memory (22), but also to one or more interfaces (11, 12, 31, 32, 33) in order to display, transmit and/or receive information. The interfaces may comprise one or more communication interfaces (11, 32, 33) and/or one or more user interfaces (12, 31). The one or more communication interfaces may be configured to send and/or receive information, for example to and/or from an MRI scanner, a CT scanner, an ultrasound camera, other computer systems, networks, data memories or the like. The one or more communication interfaces may be configured to transmit and/or receive information via physical (wired) and/or wireless communication connections. The one or more communication interfaces may comprise one or more interfaces for connection to a network, for example using technologies such as cellphone, Wi-Fi, satellite, cable, DSL, optical fiber and/or the like. In some examples, the one or more communication interfaces may comprise one or more close-range communication interfaces configured to connect devices with close-range communication technologies such as NFC, RFID, Bluetooth, Bluetooth LE, ZigBee, infrared (e.g. IrDA) or the like.

The user interfaces may include a display (31). A display (31) may be configured to display information to a user. Suitable examples thereof are a liquid crystal display (LCD), a light-emitting diode display (LED), a plasma display panel (PDP) or the like. The user input interface(s) (11, 12) may be wired or wireless and may be configured to receive information from a user in the computer system (1), for example for processing, storage and/or display. Suitable examples of user input interfaces are a microphone, an image or video recording device (for example a camera), a keyboard or a keypad, a joystick, a touch-sensitive surface (separate from a touchscreen or integrated therein) or the like. In some examples, the user interfaces may contain an automatic identification and data capture technology (AIDC) for machine-readable information. This can include barcodes, radiofrequency identification (RFID), magnetic strips, optical character recognition (OCR), integrated circuit cards (ICC) and the like. The user interfaces may further include one or more interfaces for communication with peripherals such as printers and the like.

One or more computer programs (40) may be stored in the memory (22) and executed by the processing unit (21), which is thereby programmed to perform the functions described in this description. The retrieving, loading and execution of instructions of the computer program (40) may take place sequentially, such that an instruction is respectively retrieved, loaded and executed. However, the retrieving, loading and/or execution may also take place in parallel.

The computer system of the present disclosure may be designed as a laptop, notebook, netbook and/or tablet PC; it may also be a component of an MRI scanner, a CT scanner or an ultrasound diagnostic device.

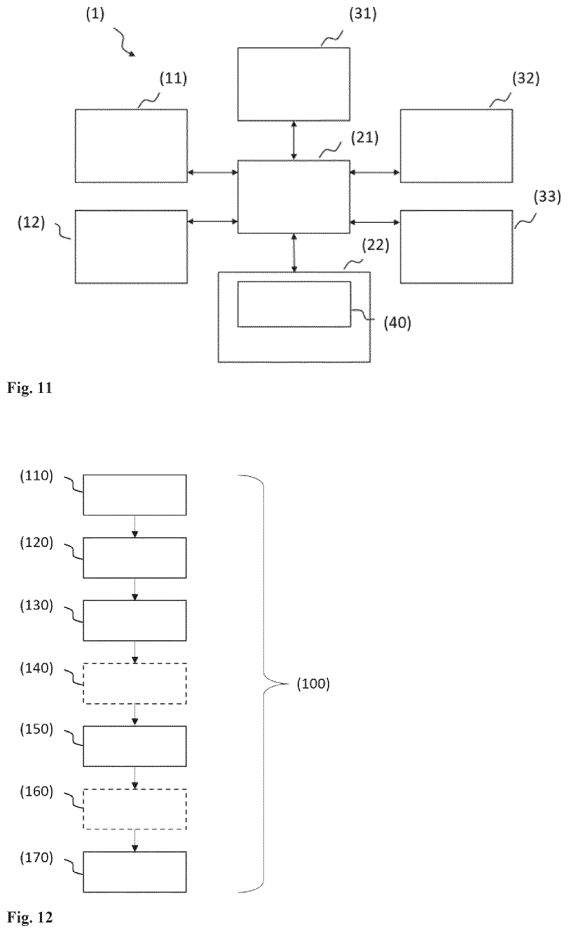

FIG. 12 shows by way of example and in schematic form an embodiment of the computer-implemented method in the form of a flowchart.

The method (100) comprises the following steps:
(110) receiving or generating a first representation, where the first representation represents an examination region of an examination object without contrast agent or after administration of a first amount of a contrast agent in real space or in frequency space,
(120) receiving or generating a second representation, where the second representation represents the examination region of the examination object after an administration of a second amount of the contrast agent in real space or in frequency space,
(130) generating a third representation based on the first representation and the second representation, where the generation of the third representation comprises a subtraction of the first representation from the second representation,
(140) optionally generating a weighted third representation by applying a frequency-dependent weight function to the third representation in frequency space,
(150) generating a fourth representation, where the generation of the fourth representation comprises an α-fold addition of the optionally weighted third representation to the first representation or second representation, where α is a positive or negative real number,
(160) if the fourth representation represents the examination region in frequency space: transforming the fourth representation into a fourth representation of the examination region in real space,
(170) outputting and/or storing the fourth representation of the examination region in real space and/or transmitting the fourth representation of the examination region in real space to a separate computer system.

The present invention can be used for various purposes. Some examples of use are described below, without the invention being intended to be limited to these examples of use.

A first example of use concerns magnetic resonance imaging examinations for differentiating intraaxial tumors such as intracerebral metastases and malignant gliomas. The infiltrative growth of these tumors makes it difficult to differentiate exactly between tumor and healthy tissue. Determining the extent of a tumor is however crucial for surgical removal. Distinguishing between tumors and healthy tissue is facilitated by administration of an extracellular; after intravenous administration of a standard dose of 0.1 mmol/kg body weight of the extracellular MRI contrast agent gadobutrol, intraaxial tumors can be differentiated much more readily. At higher doses, the contrast between lesion and healthy brain tissue is increased further; the detection rate of brain metastases increases linearly with the dose of the contrast agent (see for example M. Hartmann et al.: Does the administration of a high dose of a paramagnetic contrast medium (Gadovist) improve the diagnostic value of magnetic resonance tomography in glioblastomas?doi: 10.1055/s-2007-1015623).

A single triple dose or a second subsequent dose may be administered here up to a total dose of 0.3 mmol/kg body weight. This exposes the patient and the environment to additional gadolinium and in the case of a second scan, incurs further additional costs.

The present invention can be used to avoid the dose of contrast agent exceeding the standard amount. A first MRI image can be generated without contrast agent or with an amount less than the standard amount and a second MRI image generated with the standard amount. On the basis of these generated MRI images it is possible, as described in this disclosure, to generate a synthetic MRI image in which the contrast between lesions and healthy tissue can be varied within wide limits by altering the gain factor α. This makes it possible to achieve contrasts that are otherwise achievable only by administering an amount of contrast agent larger than the standard amount.

Another example of use concerns the reduction of the amount of MRI contrast agent in a magnetic resonance imaging examination. Gadolinium-containing contrast agents such as gadobutrol are used for a multitude of examinations. They are used for contrast enhancement in skull examinations, spine examinations, breast examinations or other examinations. In the central nervous system, gadobutrol emphasizes regions where the blood-brain barrier is impaired and/or vessels are abnormal. In breast tissue, gadobutrol visualizes the presence and extent of malignant breast disease. Gadobutrol is also used in contrast-enhanced magnetic resonance angiography for diagnosing stroke, for detecting tumor blood perfusion, and for detecting focal cerebral ischemia.

Owing to the increasing impact on the environment, to the cost burden falling on healthcare systems, and to concerns about acute side effects and possible long-term health risks, especially in the case of repeated and long-term exposure, a reduction in the dose of gadolinium-containing contrast agents is desired. This can be achieved by the present invention.

A first MRI image without contrast agent and a second MRI image with an amount of contrast agent less than the standard amount can be generated. On the basis of these generated MRI images it is possible, as described in this disclosure, to generate a synthetic MRI image in which the contrast can be varied within wide limits by altering the gain factor α. This makes it possible with less than the standard amount of contrast agent to achieve the same contrast as is obtained after administration of the standard amount.

Another example of use concerns the detection, identification and/or characterization of lesions in the liver with the aid of a hepatobiliary contrast agent such as Primovist®.

Primovist® is administered intravenously (i.v.) at a standard dose of 0.025 mmol/kg body weight. This standard dose is lower than the standard dose of 0.1 mmol/kg body weight in the case of extracellular MRI contrast agents. Unlike in contrast-enhanced MRI with extracellular gadolinium-containing contrast agents, Primovist® permits dynamic multiphase T1w imaging. However, the lower dose of Primovist® and the observation of transient motion artefacts that can occur shortly after intravenous administration means that contrast enhancement with Primovist® in the arterial phase is perceived by radiologists as poorer than contrast enhancement with extracellular MRI contrast agents. The assessment of the contrast enhancement in the arterial phase and of the vascularity of focal liver lesions is however of critical importance for accurate characterization of the lesion.

With the aid of the present invention it is possible to increase contrast, particularly in the arterial phase, without the need to administer a higher dose.

A first MRI image without contrast agent and a second MRI image during the arterial phase after administering an amount of a contrast agent that corresponds to the standard amount can be generated. On the basis of these generated MRI images it is possible, as described in this disclosure, to generate a synthetic MRI image in which the contrast in the arterial phase can be varied within wide limits by altering the gain factor α. This makes it possible to achieve contrasts that are otherwise achievable only by administering an amount of contrast agent larger than the standard amount.

Another example of use concerns the use of MRI contrast agents in computed tomography examinations.

In a CT examination, MRI contrast agents usually have a lower contrast-enhancing effect than CT contrast agents. However, it can be advantageous to employ an MRI contrast agent in a CT examination. An example is a minimally invasive intervention in the liver of a patient, where a surgeon is monitoring the procedure by means of a CT scanner. Computed tomography (CT) has the advantage over magnetic resonance imaging that more major surgical interventions are possible in the examination region while generating CT images of an examination region of an examination object. On the other hand, there are only few surgical instruments and surgical devices that are MRI-compatible. Moreover, access to the patient is restricted by the magnets used in MRI. Thus, while performing a procedure in the examination region, a surgeon will be able to visualize the examination region by CT and to follow the procedure on a monitor.

For example, if a surgeon wishes to perform a procedure in a patient's liver in order for example to carry out a biopsy on a liver lesion or to remove a tumor, the contrast between a liver lesion or tumor and healthy liver tissue will not be as pronounced in a CT image of the liver as it is in an MRI image after administration of a hepatobiliary contrast agent. There are currently no known and/or authorized CT-specific hepatobiliary contrast agents in CT. The use of an MRI contrast agent, more particularly a hepatobiliary MRI contrast agent, in computed tomography thus combines the possibility of differentiating between healthy and diseased liver tissue and the possibility of carrying out an operation with simultaneous visualization of the liver.

The comparatively low contrast enhancement achieved by the MRI contrast agent can be increased with the aid of the present invention without the need to administer a dose higher than the standard dose.

A first CT image without MRI contrast agent and a second CT image after administering an amount of an MRI contrast agent that corresponds to the standard amount can be generated. On the basis of these generated CT images it is possible, as described in this disclosure, to generate a synthetic CT image in which the contrast produced by the MRI contrast agent can be varied within wide limits by altering the gain factor α. This makes it possible to achieve contrasts that are otherwise achievable only by administering an amount of MRI contrast agent larger than the standard amount.

1. A computer-implemented method comprising:
receiving or generating a first representation, wherein the first representation represents an examination region of an examination object without contrast agent or after administration of a first amount of a contrast agent in real space or in frequency space;
receiving or generating a second representation, wherein the second representation represents the examination region of the examination object after an administration of a second amount of the contrast agent in real space or in frequency space;
generating a third representation, wherein the generation of the third representation comprises a subtraction of the first representation from the second representation;
generating a weighted third representation, wherein the generation of the weighted third representation comprises a frequency-dependent weighting of the third representation;
generating a fourth representation, wherein the generation of the fourth representation comprises an α-fold addition of the weighted third representation to the first representation or to the second representation, wherein α is a positive or negative real number;
if the fourth representation represents the examination region in frequency space: transforming the fourth representation into a fourth representation of the examination region in real space; and
outputting and/or storing the fourth representation of the examination region in real space and/or transmitting the fourth representation of the examination region in real space to a separate computer system.

2. (canceled)

3. The method as claimed in claim 1, wherein the examination object is a human.

4. The method as claimed in claim 1, wherein the examination region includes a liver, kidney, heart, lung, brain, stomach, bladder, prostate, intestine and/or a part thereof and/or another/further part of a body of a human.

5. The method as claimed in claim 1, further comprising:
receiving a first real-space representation, wherein the first real-space representation represents the examination region of the examination object without contrast agent or after administration of the first amount of the contrast agent in real space;
transforming the first real-space representation into the first representation of the examination region of the examination object in frequency space;
receiving a second real-space representation, wherein the second real-space representation represents the examination region of the examination object after administration of the second amount of the contrast agent in real space; and
transforming the second real-space representation into the second representation of the examination region of the examination object in frequency space.

6. The method as claimed in claim 1, wherein a is greater than 1.

7. The method as claimed in claim 1, wherein a is greater than 0 and less than 1.

8. The method as claimed in claim 1, wherein a is less than 0.

9. The method as claimed in claim 1, wherein the frequency-dependent weighting multiplies amplitude values of low frequencies by a larger weight factor than amplitude values of higher frequencies.

10. The method as claimed in claim 1, wherein the frequency-dependent weighting comprises a multiplication of the third representation in frequency space by a frequency-dependent weight function, wherein the frequency-dependent weight function is a Gaussian distribution function, a Hann function or a Poisson function.

11. The method as claimed in claim 1, further comprising:
receiving one or more values for a from a user.

12. The method as claimed in claim 1, further comprising:
receiving a first tonal value of a first image element of a real-space depiction of the first representation or of a real-space depiction of the second representation;
receiving a second tonal value of a second image element of a real-space depiction of the first representation or of a real-space depiction of the second representation; and
determining a value for a for which a difference between the first tonal value and the second tonal value assumes a predefined value or is above or below a predefined threshold value.

13. The method as claimed in claim 12, further comprising:
receiving a highlighting of an area within the real-space depiction of the second representation;
setting the tonal values of the area in the real-space depiction of the second representation to zero, thereby generating a modified second real-space depiction; and
generating the second representation in frequency space from the modified second real-space depiction,
wherein the generation of the third representation comprises a subtraction of the first representation from the second representation in frequency space or from the modified second real-space depiction.

14. The method as claimed in claim 12, further comprising:
for all image elements of the first real-space depiction (RIP) of the first representation: determining the first tonal value;
for all image elements of the second real-space depiction of the second representation: determining the second tonal value;
for all corresponding image elements of the first real-space depiction and the second real-space depiction: determining a quotient of the second tonal value and first tonal value;
setting to zero those tonal values of the second real-space depiction for which the quotient is greater than a predefined threshold value, thereby generating a modified second real-space depiction; and
generating the second representation from the modified second real-space depiction.

15. The method as claimed in claim 1, wherein the first representation and the second representation are the result of a magnetic resonance imaging examination and/or have been generated from magnetic resonance images.

16. The method as claimed in claim 1, wherein the first representation and the second representation are the result of a computed tomography examination and/or have been generated from computed tomography images.

17. The method as claimed in claim 1, wherein the contrast agent is an MRI contrast agent.

18. The method as claimed in claim 1, wherein the contrast agent comprises:
a Gd³⁺ complex of a compound of formula (I) 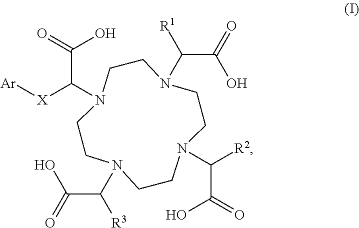
wherein:
Ar is a group selected from: 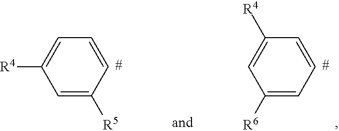
wherein # is a linkage to X;
X is a group selected from:
CH₂, (CH₂)₂, (CH₂)₃, (CH₂)₄ and *—(CH₂)₂—O—CH₂—#;
wherein * is a linkage to Ar and # is a linkage to an acetic acid residue;
R¹, R² and R³ are each independently a hydrogen atom or a group selected from C₁-C₃ alkyl, —CH₂OH, —(CH₂)₂OH and —CH₂OCH₃;
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—;
R⁵ is a hydrogen atom;
and
R⁶ is a hydrogen atom;
or a stereoisomer, a tautomer, a hydrate, a solvate or a salt thereof, or a mixture thereof, or
a Gd³⁺ complex of a compound of formula (II) 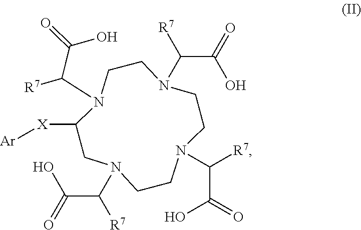
wherein:
Ar is a group selected from: 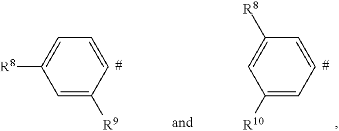
wherein # is a linkage to X;
X is a group selected from CH₂, (CH₂)₂, (CH₂)₃, (CH₂)₄ and *—(CH₂)₂—O—CH₂—#;
wherein * is a linkage to Ar and # is a linkage to the acetic acid residue;
R⁷ is a hydrogen atom or a group selected from C₁-C₃ alkyl, —CH₂OH,
—(CH₂)₂OH and —CH₂OCH₃;
R⁸ is a group selected from:
C₂-C₄ alkoxy, (H₃C—CH₂O)—(CH₂)₂—O—, (H₃C—CH₂O)—(CH₂)₂—O—(CH₂)₂—O— and
(H₃C—CH₂O)—(CH₂)₂—O—(CH₂)₂—O—(CH₂)₂—O—;
R⁹ and R¹⁰ are each independently a hydrogen atom;
or a stereoisomer, a tautomer, a hydrate, a solvate or a salt thereof, or a mixture thereof, or
the contrast agent comprises one of the following substances:
gadolinium (III) 2-[4,7,10-tris(carboxymethyl)-1,4,7,10-tetrazacyclododec-1-yl]acetic acid;
gadolinium (III) ethoxybenzyldiethylenetriaminepentaacetic acid;
gadolinium (III) 2-[3,9-bis[1-carboxylato-4-(2,3-dihydroxypropylamino)-4-oxobutyl]-3,6,9,15-tetrazabicyclo[9.3.1]pentadeca-1 (15),11,13-trien-6-yl]-5-(2,3-dihydroxypropylamino)-5-oxopentanoate;
dihydrogen [(±)-4-carboxy-5,8,11-tris(carboxymethyl)-1-phenyl-2-oxa-5,8,11-triazatridecan-13-oato (5-)]gadolinate (2-);
tetragadolinium [4,10-bis(carboxylatomethyl)-7-{3,6,12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino) acetyl]-amino}methyl)-4,7,11,14-tetraazahepta-decan-2-yl}-1,4,7,10-tetraazacyclododecan-1-ylacetate;
gadolinium 2,2′,2″-(10-{1-carboxy-2-[2-(4-ethoxyphenyl) ethoxy]ethyl}-1,4,7,10-tetraazacyclododecane-1,4,7-triyl)triacetate;
gadolinium 2,2′,2″-{10-[1-carboxy-2-{4-[2-(2-ethoxyethoxy) ethoxy]phenyl}ethyl]-1,4,7,10-tetraazacyclododecane-1,4,7-triyl}triacetate;
gadolinium 2,2′,2″-{10-[(1R)-1-carboxy-2-{4-[2-(2-ethoxyethoxy) ethoxy]phenyl}ethyl]-1,4,7,10-tetraazacyclododecane-1,4,7-triyl}triacetate;
gadolinium (2S,2'S,2″S)-2,2′,2″-{10-[(1S)-1-carboxy-4-{4-[2-(2-ethoxyethoxy) ethoxy]phenyl}butyl]-1,4,7,10-tetraazacyclododecane-1,4,7-triyl}tris(3-hydroxypropanoate);
gadolinium 2,2′,2″-{10-[(1S)-4-(4-butoxyphenyl)-1-carboxybutyl]-1,4,7,10-tetraazacyclododecane-1,4,7-triyl}triacetate;
gadolinium (III) 5,8-bis(carboxylatomethyl)-2-[2-(methylamino)-2-oxoethyl]-10-oxo-2,5,8,11-tetraazadodecane-1-carboxylate hydrate;
gadolinium (III) 2-[4-(2-hydroxypropyl)-7,10-bis(2-oxido-2-oxoethyl)-1,4,7,10-tetrazacyclododec-1-yl]acetate;
gadolinium (III) 2,2′,2″-(10-((2R,3S)-1,3,4-trihydroxybutan-2-yl)-1,4,7,10-tetraazacyclododecane-1,4,7-triyl)triacetate;
gadolinium 2,2′,2″-{(2S)-10-(carboxymethyl)-2-[4-(2-ethoxyethoxy)benzyl]-1,4,7,10-tetraazacyclododecane-1,4,7-triyl}triacetate;
gadolinium 2,2′,2″-[10-(carboxymethyl)-2-(4-ethoxybenzyl)-1,4,7,10-tetraazacyclododecane-1,4,7-triyl]triacetate.

19. A computer system comprising:
a receiving unit;
a control and calculation unit; and
an output unit,
wherein the control and calculation unit is configured to:
to cause the receiving unit to receive a first representation or to generate the first representation, wherein the first representation represents an examination region of an examination object without contrast agent or after administration of an initial amount of a contrast agent in real space or in frequency space;
cause the receiving unit to receive a second representation, wherein the second representation represents the examination region of the examination object after an administration of a second amount of the contrast agent in real space or in frequency space;
generate a third representation based on the first representation and the second representation, wherein the generation of the third representation comprises a subtraction of the first representation from the second representation;
on the basis of the third representation, generate a weighted third representation by frequency-dependent weighting of the third representation;
generate a fourth representation, wherein the generation of the fourth representation comprises an α-fold addition of the third representation to the first representation or to the second representation, wherein α is a positive or negative real number;
transform the fourth representation into a representation of the examination region in real space, when the fourth representation represents the examination region in frequency space; and
cause the output unit to output the fourth representation of the examination region in real space and/or to store the fourth representation and/or to transmit the fourth representation to a separate computer system.

20. A computer program product comprising a data carrier on which is stored a computer program that can be loaded into a working memory of a computer system, wherein the computer program causes the computer system to execute the following steps:
receive or generate a first representation, wherein the first representation represents an examination region of an examination object without contrast agent or after administration of a first amount of a contrast agent in real space or in frequency space;
receive or generate a second representation, wherein the second representation represents the examination region of the examination object after an administration of a second amount of the contrast agent in real space or in frequency space;
generate a third representation, wherein the generation of the third representation comprises a subtraction of the first representation from the second representation;
generate a weighted third representation, wherein the generation of the weighted third representation comprises a frequency-dependent weighting of the third representation;
generate a fourth representation, wherein the generation of the fourth representation comprises an α-fold addition of the optionally-weighted third representation to the first representation or to the second representation, wherein α is a positive or negative real number;
if the fourth representation represents the examination region in frequency space: transform the fourth representation into a fourth representation of the examination region in real space; and
output and/or store the fourth representation of the examination region in real space and/or transmit the fourth representation of the examination region in real space to a separate computer system.

21-25. (canceled)
